(19) United States
(12) Patent Application Publication
(10) Pub. No.: US 2003/0149278 A1
(43) Pub. Date: Aug. 7, 2003

(54) Method for preparing (+)-biotine

(76) Inventors: Michael Schwarz, Weiterstadt (DE); Ulrich Heywang, Darmstadt (DE); Thomas Koppe, Schaffhausen (CH)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/332,232

(22) PCT Filed: Jun. 20, 2001
(86) PCT No.: PCT/EP01/06965

(30) Foreign Application Priority Data
Jul. 7, 2000 (DE) 100332846

(51) Int. Cl. C07D 235/02

(52) U.S. Cl. 548/303.7

(57) ABSTRACT

The invention relates to a novel method for preparing biotine while using 5-hydroxy-2(5H)-furanone as a starting substance. The invention also relates to a method in which 1-chlorosulfonyl-1,2,2a,3,5,5a-hexa-hydro-3-((1R)-menthyloxy-)-furo[3,4-b]azet-2-one is formed as an intermediate product.

The invention relates to a novel process for the preparation of (+)-biotin using 5-hydroxy-2(5H)-furanone as starting material.

(+)-Biotin is known as vitamin H. However, it is also employed as pharmaceutical active substance for the treatment of dermatosis and as feed additive with a growth-promoting action for production animals.

Various processes have been described for the preparation of (+)-biotin.

U.S. Pat. No. 2,489,232 discloses a process by which racemic biotin is prepared. Since, however, as is known, only optically active (+)-biotin is biologically active, the racemic biotin prepared in this way subsequently had to be separated into the optical antipodes. On the one hand, all reaction steps here are carried out with racemic material, meaning that twice the amount of substances have to be processed. On the other hand, the resolution of racemic biotin into the corresponding antipodes is a fairly complicated process which in addition is also uneconomic since the undesired antipode is virtually impossible to racemise and cannot be recycled into the process.

An improvement to this process is disclosed in U.S. Pat. No. 2,489,235, where the racemate resolution is now carried out at an earlier stage. However, this process likewise still has the disadvantage that most reaction steps are carried out with racemic material and that, here too, the undesired antipode formed in this resolution is likewise virtually impossible to racemise and cannot be recycled into the process.

M. Murakami and co-workers have developed an improved process for the preparation of dl-biotin (cf. Japanese Patents 31 669/1970, 37 775/1970, 37 776/1970 and 3 580/1971). The improvement consists in introducing a carboxybutyl group into the 4-position of the dl-1,3-dibenzylhexahydrothieno[3,4-d]imidazole-2,4-dione. This dione is reacted with a 1,4-dihalobutylmagnesium and subsequently carboxylated using carbon dioxide.

A further process improvement has been described by Gerecke et al. in German Patent 20 58 248, in which an optically active lactone is prepared as optically active intermediate at an earlier stage by optical resolution of a triethylamine salt or of an ephedrine salt and by reaction with alkali metal borohydrides.

A disadvantage which is significant for industrial application consists in the use of the expensive, optically active compounds cholesterol or ephedrine and the expensive alkali metal borohydrides. The processes of EP applications 0 161 580 and 0 173 185 are afflicted with the same disadvantage, namely the use of expensive optically active compounds.

In addition, EP application 0 154 225 discloses the preparation of biotin from 1,3-dibenzylhexahydro-1H-thienoimidazolediones via a special Grignard reaction with a trioxaadamantylbutylmagnesium bromide, further dehydration and removal of the corresponding protecting groups. This process is just as unsuitable for an industrial process, especially owing to its expensive Grignard compound.

The object of the invention was therefore to provide a simple process for the preparation of (+)-biotin which results in the desired product in high yields starting from an inexpensive, readily accessible compound in the fewest possible steps which are straightforward to carry out.

The object is achieved by a novel process for the preparation of (+)-biotin using 5-hydroxy-2(5H)-furanone as starting material. The object is furthermore achieved by a process in which 1-chlorosulfonyl-1,2,2a,3,5,5a-hexahydro-3-((1R)-menthyloxy)furo[3,4-d]azet-2-one is formed as intermediate.

It is particularly preferred here for the 1-chlorosulfonyl-1,2,2a,3,5,5a-hexahydro-3-((1R)-menthyloxy)furo[3,4-d]azet-2-one to be converted into 2,3,3a,4,5,5a-hexahydro-5-oxofuro[3,4-d]imidazol-2-one with ring opening by nucleophilic attack, rearrangement, cyclisation, reduction and hydrolysis. This product can then be converted further into (+)-biotin in a known manner, if desired with introduction of protecting groups.

The ring opening can be induced in accordance with the invention by various nucleophiles. The choice of nucleophile influences the further course of the process, in particular the rearrangement, as far as the formation of the 2,3,3a,4,5,5a-hexahydro-5-oxofuro[3,4-d]imidazol-2-one. For the purposes of the invention, preferred nucleophiles are azides, in particular sodium azide, ammonia and hydroxylamine.

In a preferred variant, the ring opening is induced by ammonia as nucleophile, and the resultant ring-opening product is converted further analogously to a Hofmann degradation. In another, likewise preferred variant, the ring opening is induced by hydroxylamine as nucleophile, and the resultant ring-opening product is converted further analogously to a Beckmann rearrangement. It is particularly preferred in accordance with the invention if the ring opening is induced by an azide, preferably sodium azide, as nucleophile, and the resultant ring-opening product is converted further by a temperature increase and subsequent reduction, preferably using disodium sulfite.

In detail, the process according to the invention is carried out as described in greater detail below.

In order to carry out the process, the 5-hydroxy-2(5H)-furanone is preferably firstly acetalated using menthol. 5-Hydroxy-2(5H)-furanone and (−)-menthol give (R)-5-((1R)-menthyloxy)-2(5H)-furanone here.

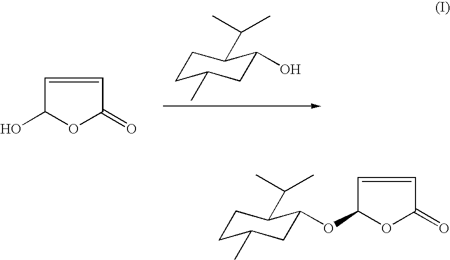

This synthetic step can be carried out by a process disclosed in the literature (Feringa, B. L.; Lange, B. de; Jong, J. C. de; J. Org. Chem. 1989, 54, 2471-2475).

In an advantageous variant, the two starting materials furanone and menthol are mixed with one another in a molar ratio of from 1:1.1 to 1:1.3, in particular 1:1.2, and heated to a temperature of from 90 to 140° C., preferably to a temperature of from about 115 to 125° C. After completion of the reaction, i.e. approximately after a reaction time of from 65 to 80 hours, preferably from about 70 to 75 hours, the unreacted menthol is distilled off. The residue obtained is subsequently purified, for example by crystallisation.

The (R)-5-((1R)-menthyloxy)-2(5H)-furanone obtained can preferably be reacted with chlorosulfonyl isocyanate analogously to a method described in the literature (Fliri, A.; Hohenlohe-Oehringen, K.; Chem. Ber. 1980, 113, 607-613; Hohenlohe-Oehringen, K. Fliri, A. [Hoffmann-LaRoche & Co AG] EP 0 022 446 (1980)) for the reaction of (2H)-chromene.

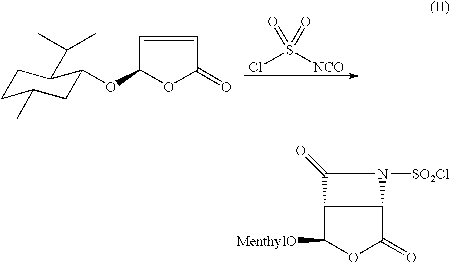

In this literature-analogous variant, chlorosulfonyl isocyanate (CSI) is introduced into a reaction flask and cooled to a temperature of from about −70 to −50° C., preferably from −65 to −55° C., in particular to −60° C. A solution of (R)-5-((1R)-menthyloxy)-2(5H)-furanone in absolute tetrahydrofuran is added dropwise with further cooling and at constant temperature and with stirring. In order to complete the reaction, the mixture is subsequently stirred for a further 12 to 20 hours at from −45 to −25° C., in particular at −35° C. In order to carry out this reaction, (R)-5-((1R)-menthyloxy)-2(5H)-furanone and chlorosulfonyl isocyanate are preferably employed in equimolar amounts.

The 1-chlorosulfonyl-1,2,2a,3,5,5a-hexahydro-3-((1R)-menthyloxy)furo[3,4-d]-azet-2-one obtained by this reaction can advantageously be employed without further purification for the further reaction.

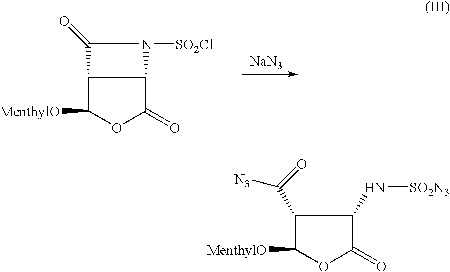

For this purpose, approximately a 1.5- to 2.5-fold amount of sodium azide is preferably dissolved in water, if desired cooled to a temperature of from −10 to 0° C. and added dropwise to the 1-chlorosulfonyl-1,2,2a,3,5,5a-hexahydro-3-((1R)-menthyloxy)furo[3,4-d]azet-2-one-containing reaction solution, which is cooled to a temperature of from −10 to 0° C. After the reaction solution obtained in this way has carefully been warmed to a temperature of from 15 to 35° C., preferably room temperature, the pH is adjusted to a value of between 6.5 and 7.5 by addition of a mineral acid from the group consisting of hydrochloric acid, nitric acid, sulfuric acid and phosphoric acid. Concentrated hydrochloric acid is preferably employed for this purpose. The bisazide formed by the reaction with sodium azide is subsequently extracted with an organic solvent from the group consisting of ether, ethyl acetate, dichloromethane, toluene and xylene, preferably with ethyl acetate.

This solution likewise requires no further purification. It can be employed directly in the next synthetic step, in which, in a particularly preferred process, elimination of nitrogen takes place through careful warming.

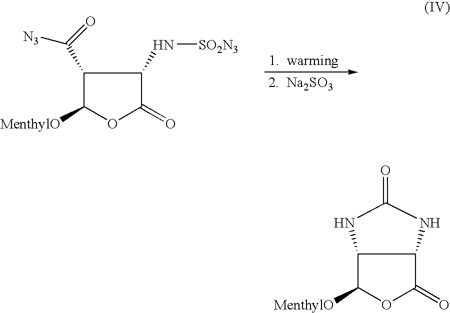

If the reaction is carried out in toluene, evolution of nitrogen commences at a temperature of about 80° C. In order to complete the reaction, the reaction solution is heated at a temperature of 75-95° C., preferably 85° C., for from about 45 minutes to two hours with stirring. During cooling, the reaction product 1-azidosulfonyl-2,3,3a,4,6,6a-hexahydro-4-((1R)-menthyloxy)-6-oxofuro[3,4]imidazol-2-one precipitates out. Hydrolysis to 2,3,3a,4,6,6a-hexahydro-4-((1R)-menthyloxy)-6-oxofuro[3,4-d]imidazol-2-one can preferably be carried out by boiling with Na₂SO₃ in water as solvent. The product then precipitates out in crystalline form on cooling of the reaction solution. Evaporation of the mother liquor under reduced pressure enables the amount of product obtained to be increased, so that a yield of from 90 to 95% can be obtained.

In a subsequent synthetic step, the menthyloxy group can be cleaved off by known methods (for example Jong, J. C. de; Feringa, B.; Tetrahedron Lett. 1989, 30, 7239-7240) and the lactone can be prepared by reduction of the hemiacetal formed as intermediate.

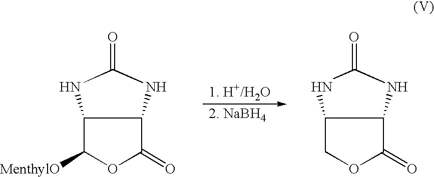

In order to carry out this reaction by the process described, 2,3,3a,4,6,6a-hexahydro-4-((1R)-menthyloxy)-6-oxofuro[3,4-d]imidazol-2-one is dissolved in a suitable solvent, such as, for example, methanol, ethanol, ethylene glycol, ethylene glycol monomethyl ether or ethylene glycol dimethyl ether, and 1 N hydrochloric acid is added. The pH of the solution is preferably adjusted to a value of between 1 and 2. The hemiacetal obtained by removal of the menthyl radical is preferably reduced to 4-hydroxymethyl-2-oxo-2,3,4,5-tetrahydroimidazole-5-carboxylic acid in the presence of NaBH₄. Under the prevailing reaction conditions, the carboxylic acid immediately cyclises in an analogous manner to that described in U.S. Pat. No. 2,489,233 (Hoffmann-LaRoche & Co AG, Goldberg, M. W.; Sternbach, L. H.) to the known 2,3,3a,4,5,5a-hexahydro-5-oxofuro[3,4-d]imidazol-2-one, which can be isolated by evaporation of the reaction solution.

The 2,3,3a,4,5,5a-hexahydro-5-oxofuro[3,4-d]imidazol-2-one obtained in this way can be protected on the two nitrogen atoms by introduction of benzyl groups analogously to the process described in EP-0 273 270 (Lonza A G; Mc Garrity, J; Tenud, L.).

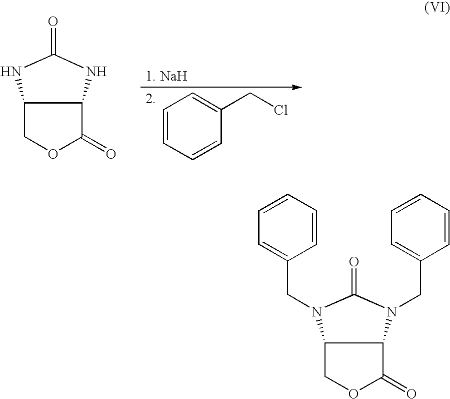

In order to carry out the benzylation, the lactone is advantageously dissolved in an aprotic solvent from the group consisting of dimethylformamide, dimethylacetamide and N-methylpyrrolidone, preferably in dimethylformamide, and approximately a 2- to 2.5-fold molar amount of benzyl chloride is added. The reaction mixture is cooled to a temperature of between −15 and 5° C., preferably between −10 and 0° C., and a 2- to 2.5-fold molar amount of NaH is added little by little in small portions with stirring. During this addition, the temperature should be maintained and should if possible not rise above 0° C., but in particular not above 5° C.

The reaction mixture is subsequently stirred for a further 1.5 to 3 hours at a temperature of from 15 to 30° C., preferably at room temperature. Acetic acid is subsequently added in an amount of from 0.1 to 0.3 mol per mol of NaH employed. The reaction solution is evaporated to dryness under reduced pressure. The residue is taken up in water and extracted with a nonpolar aprotic solvent from the group consisting of toluene, xylene, hexane and heptane, preferably with toluene. The organic phase is evaporated again, and the crude product obtained as residue is recrystallised with an alcohol, preferably with ethanol.

The dibenzylated lactone can then be converted into (+)-biotin via the thiolactone in a known manner, as described, for example, in U.S. Pat. No. 3,740,416.

The 1-chlorosulfonyl-1,2,2a,3,5,5a-hexahydro-3-((1R)-menthyloxy)furo[3,4-d]-azet-2-one obtained as intermediate does not, however, only lead to (+)-biotin by this route. Besides the above-described ring opening with the azide, other nucleophiles are also suitable for this purpose, consequently resulting in correspondingly other intermediates.

The ring-opening products obtained with the aid of other nucleophiles, such as, preferably, ammonia or hydroxylamine, can, as shown in reaction scheme VIII, likewise be converted into 2,3,3a,4,5,5a-hexahydro-5-oxofuro[3,4-d]imidazol-2-one as biotin precursor by methods known per se.

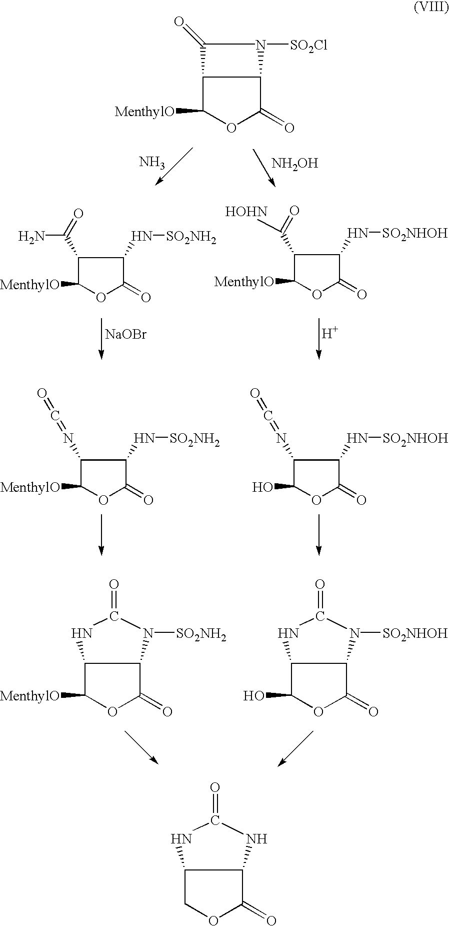

The intermediate here is converted further in a preferred variant analogously to a Hofmann degradation (Donaruma, L. G.; Heldt, W. Z.; Org. Reactions 11, 1 (1960)). The oxidation of the acid amide formed by nucleophilic ring opening with ammonia can be induced here using Br₂ or Cl₂, but also, for example, using sodium hypobromite, which is easier to handle. The isocyanate formed as intermediate then cyclises with the azidosulfonyl function present in the molecule. The subsequent reduction to 2,3,3a,4,5,5a-hexahydro-5-oxofuro[3,4-d]imidazol-2-one can then be carried out as described above.

Another process variant which is likewise preferred in accordance with the invention utilises the reaction known as the Beckmann rearrangement (Wallis, E. S.; Org. Reactions 3, 267(1946)) for the formation of 2,3,3a,4,5,5a-hexahydro-5-oxofuro[3,4-d]imidazol-2-one. The oxime formed by nucleophilic ring opening with hydroxylamine is likewise converted into an isocyanate as intermediate. The rearrangement can be induced by acids, such as phosphoric acid or sulfuric acid, or by phosgene, phosphorus oxychloride, phosphorus pentachloride or phosphorus pentoxide. As a side-reaction in the rearrangement in acids, the menthyl ether protecting group is removed directly. The isocyanate in turn cyclises with the azidosulfonyl function present in the molecule. The subsequent reduction to 2,3,3a,4,5,5a-hexahydro-5-oxofuro[3,4-d]imidazol-2-one can likewise be carried out as already described above.

The following examples serve to explain the present invention without restricting its scope of protection.

### EXAMPLE 1

### Preparation of (R)-5-((1R)-menthyloxy-2(5H)-furanone

50 mmol of 5-hydroxy-2(5H)-furanone and 60 mmol of (−)-menthol are mixed with one another and heated to 120° C. with stirring. After a reaction time of 72 hours, excess menthol is distilled off. The resultant residue is distilled under reduced pressure and subsequently recrystallised from petroleum ether, giving enantiomerically pure (R)-5-((1R)-menthyloxy)-2(5H)-furanone. The further product is isolated from the mother liquor by acidic epimerisation and further crystallisation.

Yield: 62% of theory

Melting point: 70-71° C.

[α]²⁰_{D}:−136.4° C. (ethanol)

### EXAMPLE 2

### Preparation of 1-azidosulfonyl-2,3,3a,4,6,6a-hexahydro-4-((1R)-menthyloxy)-6-oxofuro[3.4-d]imidazol-2-one

50 mmol of chlorosulfonyl isocyanate (CSI) are introduced into a reaction flask and cooled to −60° C. A solution consisting of 50 mmol of (R)-5-((1R)-menthyloxy)-2(5H)-furanone and absolute tetrahydrofuran is added dropwise at constant temperature with stirring. In order to complete the reaction, the mixture is subsequently stirred for a further 16 hours at −35° C.

The 1-chlorosulfonyl-1,2,2a,3,5,5a-hexahydro-3-((1R)-menthyloxy)furo[3,4-d]-azet-2-one obtained in this way is not isolated, but instead immediately converted further.

To this end, a solution of sodium azide (100 mmol) in water is carefully added dropwise to the 1-chlorosulfonyl-1,2,2a,3,5,5a-hexahydro-3-((1R)-menthyloxy)-furo[3,4-d]azet-2-one at from −10° C. to 0° C. When the addition is complete, the mixture is carefully warmed to RT, and the pH of the solution is adjusted to a value in the range from 6.5 to 7.5 using conc. hydrochloric acid. The bisazide formed in this way is extracted with toluene.

The toluene phase, which contains the bisazide, is slowly warmed to about 85° C. Nitrogen evolution commences from about 80° C. The mixture is warmed with stirring for approximately a further 1 hour until the reaction is complete and gas evolution no longer occurs. On cooling, 1-azidosulfonyl-2,3,3a,4,6,6a-hexahydro-4-((1R)-menthyloxy)-6-oxofuro[3,4-d]imidazol-2-one precipitates out.

Yield: 52% of theory

Characterisation: spectroscopic data correspond to the literature values.

### EXAMPLE 3

### Preparation of 2,3,3a,4,6,6a-hexahydro-4-((1R)-menthyloxy)-6-oxofuro[3,4-d]imidazol-2-one

The product from Example 2 is heated at the boil for 5 hours with 7 g of Na₂SO₃ in 750 ml of water. When the reduction is complete, crystalline 2,3,3a,4,6,6a-hexahydro-4-((1R)-menthyloxy)-6-oxofuro[3,4-d]imidazol-2-one precipitates out on cooling. Further fractions are obtained by evaporation of the mother liquor.

Yield: 92% of theory

Characterisation: spectroscopic data correspond to the literature values.

### EXAMPLE 4

### Preparation of 2,3,3a,4,5,5a-hexahydro-5-oxofuro[3,4-d]imidazol-2-one

2,3,3a,4,6,6a-Hexahydro-4-((1R)-menthyloxy)-6-oxofuro[3,4-d]imidazol-2-one is dissolved in acetone, and 1 N HCl is added. The hemiacetal formed on removal of the menthyl radical is reduced to 4-hydroxymethyl-2-oxo-2,3,4,5-tetrahydro-imidazole-5-carboxylic acid using NaBH₄. The open compound cyclises under the reaction conditions to give the known 2,3,3a,4,5,5a-hexahydro-5-oxofuro[3,4-d]-imidazol-2-one, which is isolated from the reaction solution by evaporation.

Yield: 85% of theory

Characterisation: melting point Tₘ=200-202° C.

### EXAMPLE 5

### Introduction of Benzyl Groups for Protection of the Lactone

The lactone (50 mmol) (product from Example 4) is dissolved in DMF, and benzyl chloride (110 mmol) is added. NaH (110 mmol) is subsequently added in small portions at from −10° C. to 0° C. After the mixture has been stirred at RT for 2 hours, acetic acid (10 mmol) is added, and the reaction solution is evaporated. The residue is taken up in water and extracted with toluene. The toluene solution is evaporated to dryness. The crude product is crystallised from ethanol.

Yield: 88% of theory

Melting point Tₘ=119-120° C.

1. Process for the preparation of (+)-biotin, characterised in that 5-hydroxy-2(5H)-furanone is employed as starting compound.

2. Process for the preparation of (+)-biotin, characterised in that 1-chlorosulfonyl-1,2,2a,3,5,5a-hexahydro-3-((1R)-menthyloxy)-furo[3,4-d]azet-2-one is formed as intermediate.

3. Process according to claim 1 and/or claim 2, characterised in that 1-chlorosulfonyl-1,2,2a,3,5,5a-hexahydro-3-((1R)-menthyloxy)furo-[3,4-d]azet-2-one is converted into 2,3,3a,4,5,5a-hexahydro-5-oxofuro[3,4-d]imidazol-2-one with ring opening by nucleophilic attack, rearrangement, cyclisation, reduction and hydrolysis, and the latter is converted further into (+)-biotin, if desired with introduction of protecting groups.

4. Process according to claim 3, characterised in that the ring opening is induced by ammonia as nucleophile, and the resultant ring-opening product is converted further analogously to a Hofmann degradation.

5. Process according to claim 3, characterised in that the ring opening is induced by hydroxylamine as nucleophile, and the resultant ring-opening product is converted further analogously to a Beckmann rearrangement.

6. Process according to claim 3, characterised in that the ring opening is induced by an azide, preferably sodium azide, as nucleophile, and the resultant ring-opening product is converted further by a temperature increase and subsequent reduction, preferably using disodium sulfite.

7. Process according to one of claims 1 to 6, characterised in that the 5-hydroxy-2(5H)-furanone is firstly etherified with menthol.

8. Use of 5-hydroxy-2(5H)-furanone as starting compound for the preparation of (+)-biotin.
